(19) United States
(12) Patent Application Publication
(10) Pub. No.: US 2007/0149990 A1
(43) Pub. Date: Jun. 28, 2007

(54) Apparatus and methods of tissue removal within a spine

(76) Inventors: Erika I. Palmer, Menlo Park, CA (US); Alberto Ruiz Cantu, San Francisco, CA (US); Lloyd Diamond, Palo Alto, CA (US); Chris Phan, San Leandro, CA (US); Derek Rothwell, Los Altos, CA (US); Meera Sankaran, Cupertino, CA (US)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/450,878

(22) Filed: Jun. 12, 2006

Related U.S. Application Data
(60) Provisional application No. 60/697,536, filed on Jul. 11, 2005.

(51) Int. Cl. A61B 17/32; A61B 17/00; A61B 10/00

(52) U.S. Cl. 606/167; 600/562; 606/170; 606/79

(57) ABSTRACT

In one embodiment, an apparatus includes an elongate body having a collapsed configuration and an expanded configuration. The elongate body has a cutting portion at a distal portion of the elongate body, and the cutting portion defines an opening in the elongate body. The distal portion of the elongate body is configured to be inserted into a nucleus of an intervertebral disc when the elongate body is in the collapsed configuration. The cutting portion of the elongate body is configured to disrupt at least a portion of tissue within the nucleus of the intervertebral disc when the elongate body is in the expanded configuration. The elongate body is configured to capture the portion of tissue within the opening of the elongate body when the elongate body is moved from the expanded configuration to the collapsed configuration while disposed within the nucleus of the intervertebral disc.

### CROSS-REFERENCE TO RELATED APPLICATION

This application claims priority to U.S. Provisional Application Ser. No. 60/697,536, entitled “Apparatus And Methods Of Tissue Removal Within A Spine,” filed Jul. 11, 2005, the disclosure of which is incorporated herein by reference in its entirety.

### BACKGROUND

The invention relates generally to medical devices and procedures, and more particularly to a medical device for use in medical procedures associated with a spine.

Known medical devices are configured to percutaneously access an intervertebral disc to perform a variety of different medical procedures. Some known medical devices are configured to remove tissue from within the interior of an intervertebral disc. Other known medical devices are configured to provide some type of cutting means to tear or loosen tissue within the intervertebral disc. There are also known medical devices configured to provide a suction or vacuum force to assist in the removal of tissue from within the intervertebral disc.

Although medical devices that provide vacuum force are known and other medical devices that provide cutting functions are known, a need exists for an improved medical device that can provide both a cutting and vacuum force within the same device.

### SUMMARY OF THE INVENTION

Apparatuses and methods for accessing a nucleus of an intervertebral disc through the annulus are disclosed herein. In one variation, an apparatus includes an elongate body having a collapsed configuration and an expanded configuration. The elongate body has a cutting portion at a distal portion of the elongate body, and the cutting portion defines an opening in the elongate body. The distal portion of the elongate body is configured to be inserted into a nucleus of an intervertebral disc when the elongate body is in the collapsed configuration. The cutting portion of the elongate body is configured to disrupt at least a portion of tissue within the nucleus of the intervertebral disc when the elongate body is in the expanded configuration. The elongate body is configured to capture the portion of tissue within the opening of the elongate body when the elongate body is moved from the expanded configuration to the collapsed configuration while disposed within the nucleus of the intervertebral disc.

### BRIEF DESCRIPTION OF THE DRAWINGS

The invention is described with reference to the accompanying drawings. In the drawings, like reference numbers indicate identical or functionally similar elements.

FIG. 1 is a schematic representation of a medical device according to an embodiment of the invention shown inserted into an intervertebral disc.

FIG. 2 is a side perspective view of a medical device according to an embodiment of the invention.

FIG. 3 is a partial cross-sectional view of the medical device shown in FIG. 2.

FIG. 4 is a side perspective view of a portion of the medical device shown in FIG. 2.

FIG. 5 is a side perspective view of another portion of the medical device shown in FIG. 2.

FIG. 6 is a plan view of a medical device according to an embodiment of the invention shown inserted into an intervertebral disc.

FIG. 7 a partial cross-sectional view of the medical device shown in FIG. 6.

FIG. 8 is a partial cross-sectional view of a medical device according to an embodiment of the invention.

FIG. 9 is a cross-sectional view of the medical device shown in FIG. 8.

FIG. 10 is a side perspective view of a portion of a medical device according to an embodiment of the invention.

FIG. 11 is a side perspective view of a portion of the medical device shown in FIG. 10.

FIG. 12 is a partial cross-sectional view of a medical device according to an embodiment of the invention.

FIG. 13 is a side view of a medical device according to an embodiment of the invention.

FIG. 14 is a side view of a medical device according to an embodiment of the invention shown in an expanded configuration.

FIG. 15 is a side view of the medical device of FIG. 14 shown in a collapsed configuration.

FIG. 16 is a side view of a medical device according to an embodiment of the invention shown in an expanded configuration.

FIG. 17 is a side view of the medical device of FIG. 16 in a collapsed configuration.

FIG. 18 is a side view of a medical device according to an embodiment of the invention.

FIG. 19 is a side perspective view of a medical device according to an embodiment of the invention.

FIG. 20 is a side view partially in cross-section of the medical device shown in FIG. 19.

FIG. 21 is a side view partially in cross-section of the medical device shown in FIG. 19.

FIG. 22 is a side view partially in cross-section of the medical device shown in FIG. 19.

### DETAILED DESCRIPTION

In one embodiment, a medical device includes an elongate body having a collapsed configuration and an expanded configuration. The elongate body has a cutting portion at a distal portion of the elongate body, and the cutting portion defines an opening in the elongate body. The distal portion of the elongate body is configured to be inserted into a nucleus of an intervertebral disc when the elongate body is in the collapsed configuration. The cutting portion of the elongate body is configured to disrupt at least a portion of tissue within the nucleus of the intervertebral disc when the elongate body is in the expanded configuration. The elongate body is configured to capture the portion of tissue within the opening of the elongate body when the elongate body is moved from the expanded configuration to the collapsed configuration while disposed within the nucleus of the intervertebral disc.

In another embodiment, an apparatus includes a cannula that defines a lumen, and an elongate body having a collapsed configuration and an expanded configuration. The elongate body is configured to be movably disposed within the lumen of the cannula when the elongate body is in the collapsed configuration. The elongate body has a cutting portion at a distal portion of the elongate body, and the cutting portion defines an opening in the elongate body. The distal portion of the cannula is configured to be inserted into an interior portion of an intervertebral disc when the elongate body is in the collapsed configuration. The cutting portion of the elongate body is configured to disrupt at least a portion of tissue within the interior of the intervertebral disc when the distal portion of the elongate body is moved outside of the lumen of the cannula and within the interior of the intervertebral disc while the elongate body is in the expanded configuration. The elongate body is configured to capture the portion of tissue within the opening of the elongate body when the elongate body is moved from the expanded configuration to the collapsed configuration.

A medical device (also referred to herein as an “apparatus”) can be used in a variety of minimally-invasive medical procedures such as disc nucleus replacement. The following description discusses the use of the medical device in an intervertebral disc procedure, but it should be understood that the medical device can be used in procedures on other areas of a body, including other tissue, such as bone structures, collagen tissues and/or soft tissue areas.

The term “cannula” is used here to mean a component of the medical device or apparatus having one or more passageways configured to receive a medical device therethrough and provide access to an interior portion of an intervertebral disc. For example, the cannula can be substantially tubular. The cannula can have a variety of different shapes and sizes, such as having a round or octagonal outer perimeter.

As used in this specification and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, the end of a medical device inserted inside the patient's body would be the distal end of the medical device, while the end of the medical device outside the patient's body would be the proximal end of the medical device.

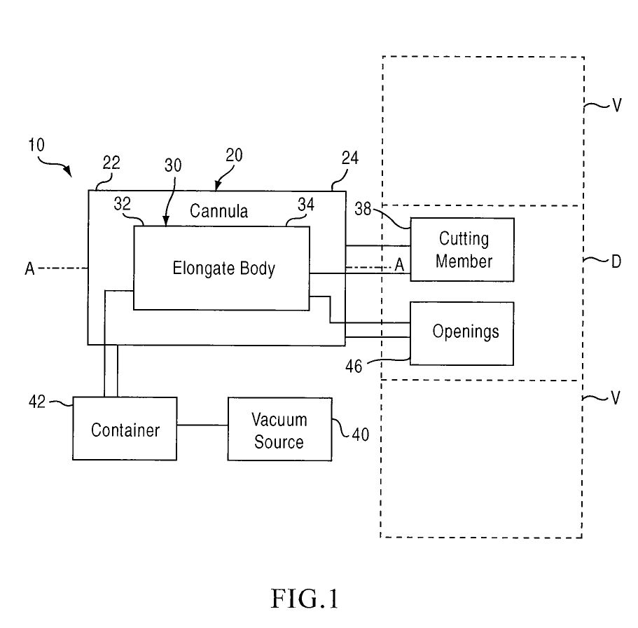

FIG. 1 is a schematic representation of a medical device according to an embodiment of the invention shown inserted within an intervertebral disc. The medical device 10 includes a cannula 20 having a proximal portion 22, a distal portion 24 and a lumen (not shown in FIG. 1) extending from the proximal portion 22 to the distal portion 24 of the cannula 20. The distal portion 24 of the cannula is configured to provide percutaneous access to an intervertebral disc D shown in FIG. 1 between two vertebral bodies V. The cannula 20 can include a sharpened distal end to penetrate the disc D or a separate device such as a stylet can be used to penetrate the disc D prior to inserting the cannula 20. An elongate body 30 can be movably disposed within the lumen of the cannula 20. The elongate body 30 includes a proximal portion 32, a distal portion 34, and an internal passageway (not shown) extending from the proximal portion 32 to the distal portion 34. The elongate body 30 can also include at least one cutting member 38 and one or more openings 46 on an outer surface of the elongate body 30. The opening(s) 46 is in communication with the internal passageway, and the internal passageway can be coupled to a suction source 40 (e.g., a vacuum source). In alternative embodiments, the cannula may include its own opening(s) and/or cutting member.

The cutting member 38 can be embodied in a variety of different manners, as will be described below, and is configured to loosen, disrupt and/or tear tissue within the interior portion (e.g., nucleus) of the intervertebral disc D. The cutting member 38 can be disposed on an outer surface of elongate body 30 or within an interior portion of elongate body 30. The cutting member 38 can also be formed on an edge or perimeter defined by one or more openings 46 on a wall of the elongate body 30. The opening(s) 46 can be located at a variety of different locations on the elongate body 30. The opening(s) 46 is configured such that tissue from within the intervertebral disc D can be pulled through the opening(s) 46 and into the internal passageway via the suction force supplied through the internal passageway 36. The tissue removed from the intervertebral disc D can be collected in a container 42 coupled to the elongate body 30. In some embodiments, the suction force can pull or draw a portion of tissue into the elongate body, and the cutting member can sever that portion of tissue from the remainder of the tissue.

In this embodiment, the elongate body 30 can move axially within the cannula 20 (e.g., along an axis A defined by a longitudinal length of the cannula 20). In other embodiments, the elongate body can rotate within the cannula. In some embodiments, the elongate body can move both rotationally and axially. A power source (not shown) can be coupled to the elongate body 30 to provide automatic movement of the elongate body 30. In some embodiments, the elongate body 30 is moved manually.

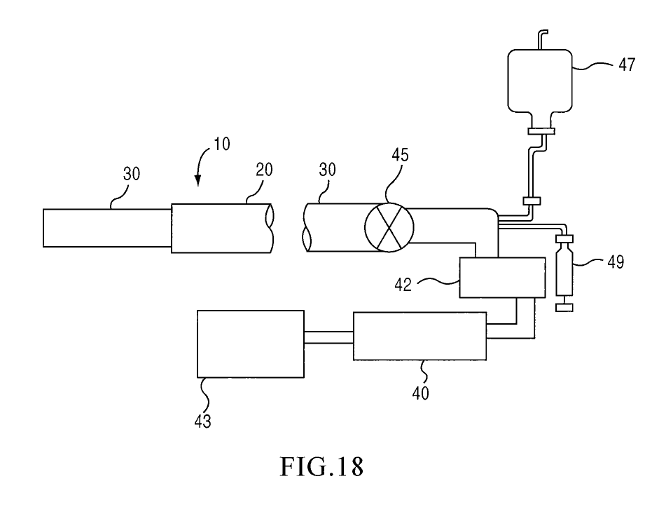

FIG. 18 illustrates various components that can be used in conjunction with the medical device of FIG. 1, and with any of the embodiments described herein. As described above, the internal passageway of the elongate body 30 is coupled to a source of suction or vacuum power 40 and a containment device, such as container 42. In addition, in some embodiments, the medical device can include irrigation components to assist with loosening the tissue within the intervertebral disc, as will be described below and/or to lubricate the transport of disrupted tissue to a location outside of the intervertebral disc. In such an embodiment, the elongate body 30 can be coupled to a source of irrigation fluid 47, providing, for example, a saline solution. The medical device can also be coupled to a power source 43 to provide power to the suction source 40, such as a battery powered pump or other suitable device. The power source 43 can provide suction power in various forms. For example, suction power can be provided in the form of a pulsing suction. The suction rate can also be varied. For example, the suction provided for removal of tissue can be provided at the same or different rate than a rate of irrigating. Thus, the suction and irrigation functions can be coordinated and configured for the particular need. In some embodiments, the source of suction power can also provide the power to actuate movement of the elongate body. A saline flush chamber 49 can also be included.

The medical device 10 can be used to remove tissue from within the interior or nucleus of the intervertebral disc D, and produce a cavity within the intervertebral disc. In some applications, the cavity is such that the resulting cavity is similar to a partial discectomy. In other applications, the cavity is larger or similar to a total discectomy. The medical device 10 can be used to produce a cavity for subsequent placement of a prosthesis (e.g., artificial nucleus) or for insertion of biocompatible material, such as a polymer, hydrogel or other nucleus replacement material. Alternatively, the medical device 10 can be used to relieve pressure from within the intervertebral disc D. For example, in some cases, tissue within an intervertebral disc can be pushed outside of the disc, through, for example, a hole in a disc annulus. In such a case, the medical device 10 can be deployed to remove tissue from within the intervertebral disc resulting in space within the disc devoid of tissue. By removing tissue from within the disc, the tissue that had previously been pushed outside of the disc may be retracted back into the disc.

Various embodiments of the medical device will now be described. It should be understood that the embodiments described herein are examples that can be implemented alone or in combination with one or more other embodiments.

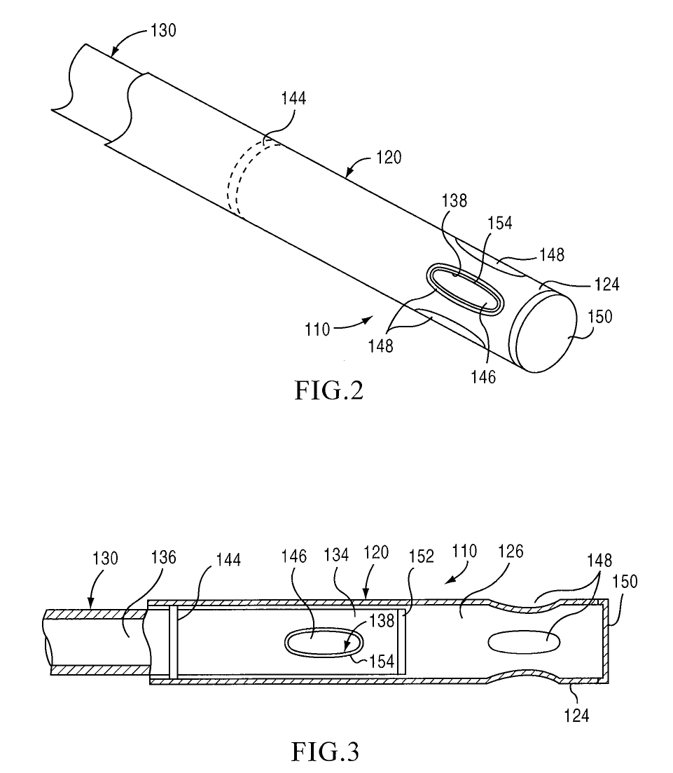

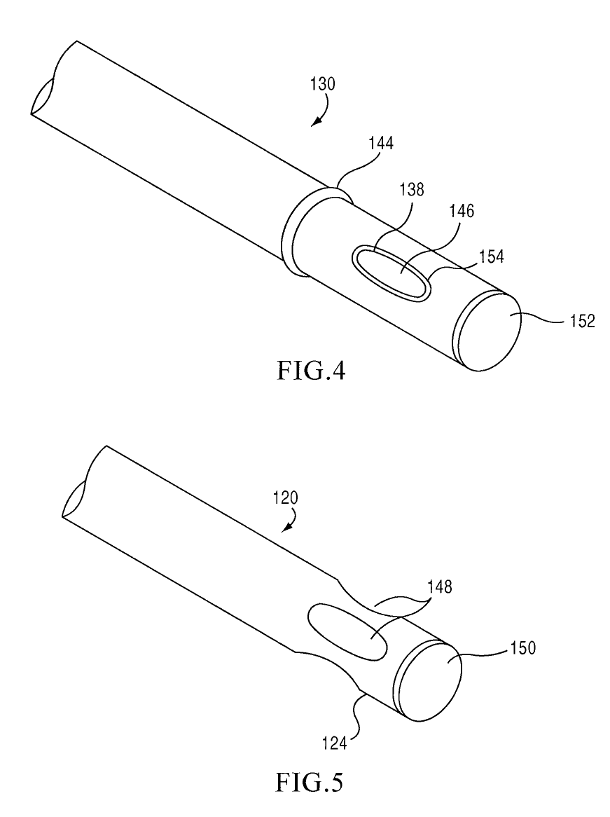

FIGS. 2-5 illustrate a portion of a medical device according to an embodiment of the invention. A medical device 110 includes a cannula 120 and an elongate body 130. The cannula 120 includes a proximal portion (not shown), a distal portion 124 and a lumen 126 extending from the proximal portion to the distal portion 124. The cannula 120 also defines at least one window 148 disposed on the distal portion 124. The at least one window 148 is in communication with the lumen 126. An end cap 150 can be disposed on a distal end of the cannula 120 to plug the distal end. Cannula 120 can alternatively include a sharp tip on the distal end instead of the end cap 150. The sharp tip can provide a means to penetrate the intervertebral disc and define an access path.

The elongate body 130 includes a proximal portion (not shown), a distal portion 134, and an internal passageway 136 (see FIG. 3) extending from the proximal portion to the distal portion 134. As with the previous embodiments, the internal passageway 136 can be coupled to a source of suction or vacuum power (not shown). The elongate body 130 also includes an opening 146 defined on the distal portion 134 and an end cap 152 disposed on a distal end of the elongate body 130. The opening 146 on the elongate body 130 includes a cutting member 138 disposed around at least a portion of a perimeter of the opening 146. The cutting member 138 can be, for example, a sharp edge formed along a portion of the perimeter of the opening 146.

The elongate body 130 can be movably disposed within the lumen 126 of the cannula 120. A seal or grommet 144 disposed on an outer surface of the elongate body 130 provides a sealing fit between the elongate body 130 and the cannula 120, while at the same time permitting the elongate body 130 to move within the lumen 126 of the cannula 120. When the distal portion 134 of the elongate body 130 is positioned within the distal portion 124 of the cannula 120, the opening 146 can be at least partially aligned with one of the at least one windows 148.

In use, the cannula 120 can be inserted into an intervertebral disc such that the distal portion 124 is positioned within an interior of the disc. The elongate body 130 can be moved to a position within the cannula 120 such that the opening 146 at least partially aligns with a selected window 148. The suction power can suction tissue from within the interior of the disc at least partially through the window 148, and at least partially through the opening 146. The elongate body 130 can be moved within the lumen 126, for example, by either rotating the elongate body 130 about its longitudinal axis or pulling the elongate body 130 in the direction of its proximal end and/or its distal end such that opening 146 and window 148 move relative to each other from being at least partially aligned to being misaligned. In either situation, the cutting member 138 on the opening 146 can sever the tissue that is at least partially positioned within the opening 146 from the remaining tissue within the disc. Once the tissue is severed, the suction force can pull the tissue through the internal passageway 136. The cutting member 138 can also be used to tear, loosen or disrupt tissue within the intervertebral disc by moving the elongate body 130 relative to the cannula 120. Because the opening 146 can be positioned such that the opening 146 is at least partially aligned with a selected one of the windows 148, tissue can be removed from various directions within the intervertebral disc without having to reposition the cannula 130. In other words, by aligning opening 146 with a given window 148 from the various windows 148 disposed about the circumference of the medical device 110, tissue can be removed from an area adjacent to a particular portion of the medical device 110 circumference.

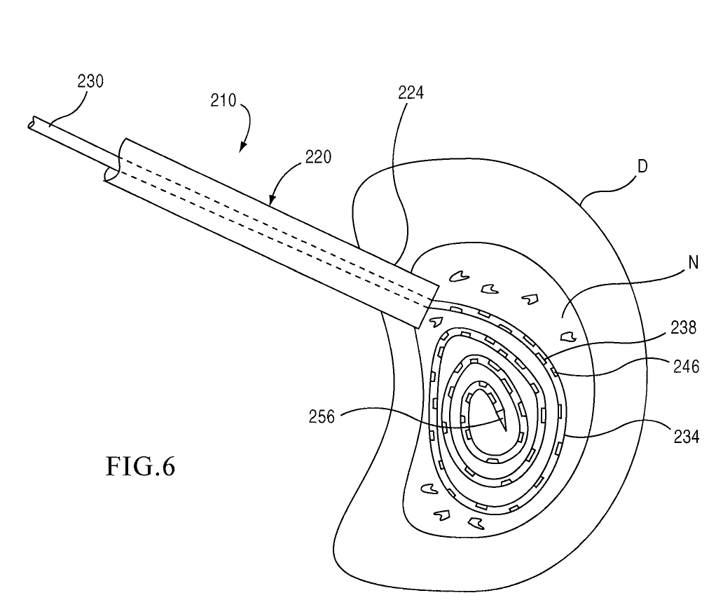

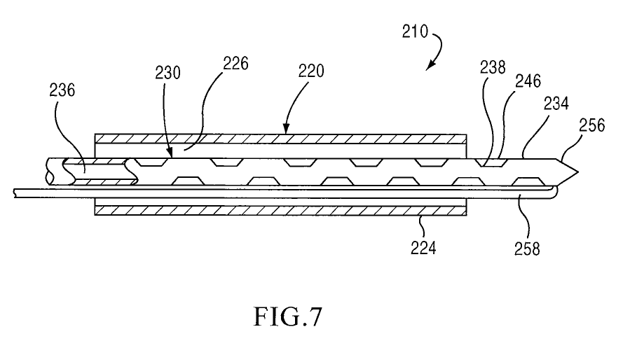

FIGS. 6 and 7 illustrate a portion of a medical device according to another embodiment of the invention. A medical device 210 includes a cannula 220 and an elongate body 230 movably disposed within a lumen 226 of the cannula 220. The cannula 220 includes a proximal portion (not shown) and a distal portion 224, and is configured to provide percutaneous access to an intervertebral disc. The elongate body 230 includes a proximal portion (not shown), a distal portion 234, and an internal passageway 236 (see FIG. 7) extending from the proximal portion to the distal portion 234. The elongate body 230 defines a plurality of openings 246 disposed along the distal portion 234 that are in communication with the internal passageway 236. In this embodiment, each of the openings 246 has a cutting member 238 disposed along at least a portion of a perimeter of the opening 246. A sharp tip 256 is disposed on a distal end of the elongate body 230. In alternative embodiments, the elongate body does not have a sharp tip disposed on a distal end.

As with the previous embodiments, the elongate body 230 can be movably disposed within the lumen 226 of the cannula 220. The internal passageway 236 can also be coupled to a source of suction or vacuum power, as previously described. In this embodiment, the elongate body 230 has a predetermined coiled configuration when the elongate body 230 is unrestrained. When the elongate body 230 is restrained, such as when the elongate body 230 is largely disposed within the lumen 226 of the cannula 220, the elongate body 230 will be in a substantially straightened configuration, as shown in FIG. 7. For example, the distal portion 234 of the elongate body 230 can comprise a shape-memory material such that the distal portion 234 can curve and form a predefined shape when deployed. In use, the distal portion 234 of the elongate body 230 can be extended distally from the cannula 220 and assume the coiled configuration, as shown in FIG. 6. As the distal portion 234 of the elongate body 230 is introduced into the nucleus N of the intervertebral disc D, the distal portion 234 begins to coil into the coiled configuration and the cutting members 238, as well as the sharp tip 256 can loosen, tear and/or disrupt tissue within the nucleus N during the coiling process. The suction force that can be provided to the internal passageway 236 can be used to suction tissue from within the nucleus N through the openings 246 and the internal passageway 236, producing a cavity within the nucleus N of the intervertebral disc D. In some applications, the distal end portion 234 of the elongate body 230 is introduced into the nucleus N multiple times. Also, the distal end portion 234 of the elongate body 230 can be configured to uncoil into various different curvatures and/or coiled configurations. In this embodiment, the coiled configuration of the distal end portion 234 can be configured to substantially fill the nucleus N, producing a cavity large enough, for example, for placement of a prosthesis within the nucleus N. In other embodiments, the distal end portion 234 of the elongate body 230 can be configured to uncoil such that it fills only a portion of the nucleus N. Also, in alternative embodiments, the medical device 220 is not coupled to a source of suction. Such an embodiment can be used, for example, when transport of tissue outside of the intervertebral disc is not desired.

Also, in some embodiments, a guide wire 258 can be coupled to the elongate body 230 to assist in straightening or uncoiling the elongate body 230 as shown in FIG. 7. In such an embodiment, the guide wire 258 can be removed from the elongate body 230 after the elongate body 230 has been inserted through the cannula 230.

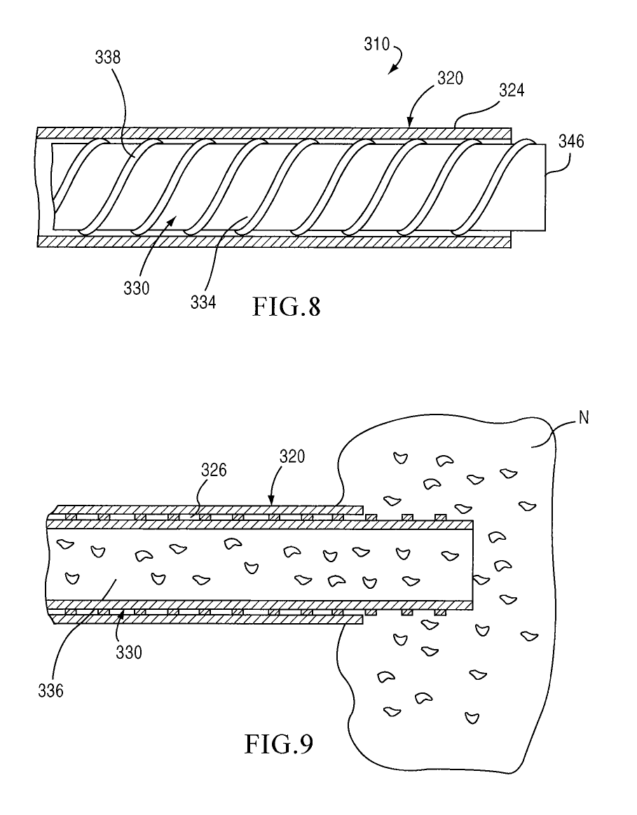

FIGS. 8 and 9 illustrate a portion of a medical device according to another embodiment of the invention. A medical device 310 includes a cannula 320 and an elongate body 330. The cannula 320 includes a proximal portion (not shown), a distal portion 324 and a lumen 326 extending from the proximal portion to the distal portion 324. In this embodiment, the cannula 320 includes an open distal end in communication with the lumen 326.

The elongate body 330 includes a proximal portion (not shown), a distal portion 334, and an internal passageway 336 (see FIG. 9) extending from the proximal portion to the distal portion 334. As with the previous embodiments, the internal passageway 336 is coupled to a source of suction or vacuum power (not shown). The elongate body 330 also includes an opening 346 defined on the distal end of the elongate body 330 in communication with the internal passageway 336. In this embodiment, the elongate body 330 includes one or more cutting members 338 disposed in a spiral configuration around an outer surface of the distal end portion 334 of elongate body 330.

The elongate body 330 can be movably disposed within the lumen 326 of the cannula 320. In use, the cannula 320 can be inserted into an intervertebral disc such that the distal portion 324 of the cannula 320 is positioned within an interior or nucleus N of the disc. The elongate body 330 can be extended axially through the lumen 326 such that at least a portion of the distal portion 334 of the elongate body 330 is extended into the nucleus N. The vacuum or suction power can suction tissue from within the nucleus N through the opening 346. For example, the elongate body 330 can be moved within the lumen 326 by either rotating the elongate body 330 or axially moving the elongate body 330. In either situation, the cutting members 338 can tear, disrupt and/or loosen tissue within the nucleus N. The suction power can be used to suction the torn, disrupted or loosened tissue through opening 346 and the internal passageway 336. In some embodiments, the suction can be performed simultaneously with the movement of the elongate body 330. In an alternative embodiment, the elongate body 330 can include multiple additional openings (not shown) proximate the cutting members 338 (i.e., along the outer surface of elongate body 330). This would allow tissue to be suctioned laterally or radially through the wall of elongate body 330.

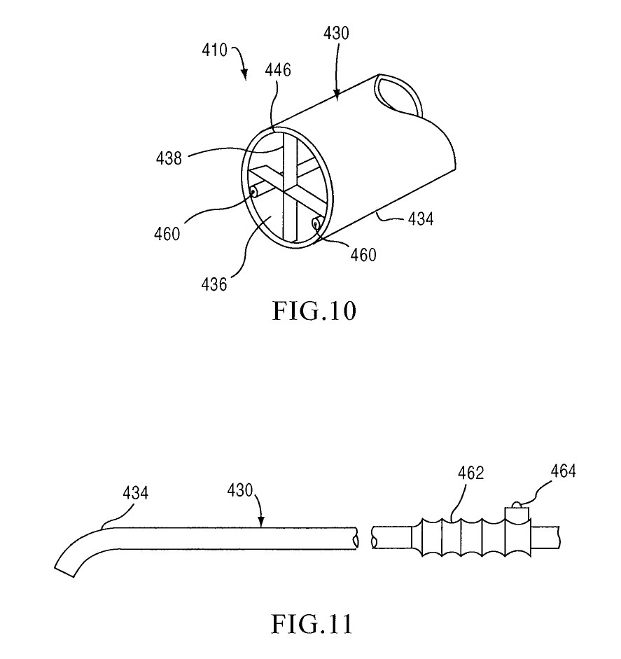

FIGS. 10 and 11 illustrate a portion of a medical device according to another embodiment of the invention. A medical device 410 includes an elongate body 430 having a proximal portion (not shown), a distal portion 434, and an internal passageway 436 extending from the proximal portion to the distal portion 434. The medical device 410 can also include a cannula (not shown) as described in any of the previous embodiments. In this embodiment, a cutting member 438 is disposed within the internal passageway 436 of the elongate body 430. In alternative embodiments, multiple cutting members 438 can be disposed within the passageway 436 of the elongate body 430. The elongate body 430 also includes a pair of irrigation lumens 460. Although this embodiment is described as having irrigation lumens 460, it is to be understood that the irrigation lumens 460 are an optional feature.

As with the previous embodiments, the internal passageway 436 can be coupled to a source of suction or vacuum power and the elongate body 430 can be moveably disposed within a lumen of a cannula (not shown). In use, the elongate body 430 can be inserted into an opening created within an intervertebral disc, or other tissue, either through a cannula or through a channel created by different device, such as a stylet. The elongate body 430 can be moved axially allowing the cutting member 438 to loosen or tear tissue within a nucleus of an intervertebral disc. Simultaneous with the cutting, the tissue can be suctioned through an opening 446 on the distal end of the elongate body 430 and through the internal passageway 436. As tissue is suctioned into the internal passageway 436, the cutting member 438 can mince the tissue and reduce the size of the tissue fragments for easier transport through the lumen 436. In some embodiments, the suction force can draw at least a portion of tissue from within the intervertebral disc and into the passageway of the elongate body. A cutting member disposed within the passageway can then sever the portion of the tissue from the remaining tissue in the disc when the elongate body is moved (e.g., rotated). In an embodiment having one or more irrigation lumens 460, an irrigation solution can be introduced into the nucleus of the intervertebral disc to help loosen tissue to be suctioned. As discussed previously, the irrigation solution can be introduced into the disc at various rates. For example, the irrigation solution can be introduced into the nucleus intermittently or in pulses, or in a continuous flow or stream.

In some embodiments, the distal end portion 434 can be configured to bend or flex as shown in FIG. 11. This provides the elongate body 430 with the ability to direct the orientation of the distal end portion 434 within the nucleus of the intervertebral disc. As shown in FIG. 11, a handle or grip 462 can be coupled to the elongate body 430. An actuation trigger 464 can be coupled to the grip 462 and configured to actuate the vacuum force and/or the irrigation function of the medical device 410. Although only shown in FIG. 11, it is to be understood that a handle and actuation trigger can be included with any of the embodiments of a medical device described herein.

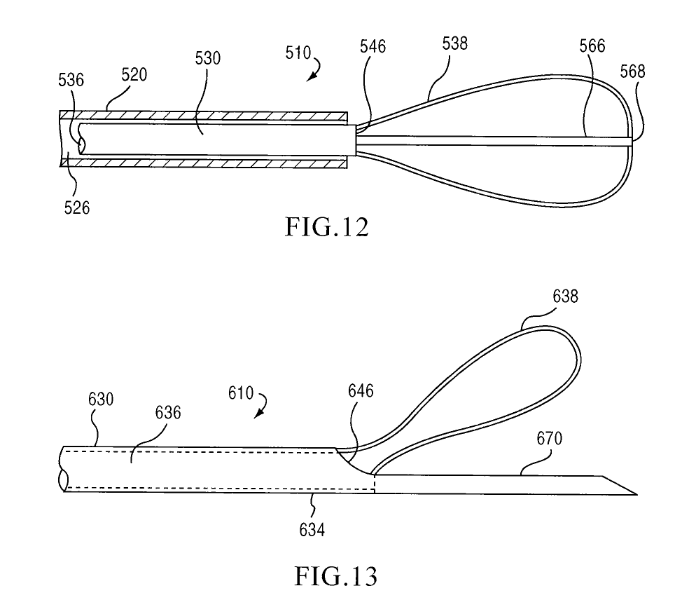

FIGS. 12 and 13 illustrate portions of additional embodiments of a medical device. These embodiments provide similar functions as the previously described embodiments. A medical device 510, illustrated in FIG. 12, includes a cannula 520 and an elongate body 530 movably disposed within a lumen 526 of the cannula 520. In this embodiment, the elongate body 530 includes a cutting member 538 in the form of a loop. The elongate body 530 includes an internal passageway 536 and an opening 546 on a distal end in communication with the internal passageway 536. The internal passageway 536 is coupled to a source of suction or vacuum power (not shown). In addition, the elongate body 530 can include an optional support 566, extending from a distal end of the elongate body 530 to a distal end of the cutting member 538. The support can optionally define a lumen (not shown) extending along its longitudinal length in communication with the internal passageway 536 and an opening 568 at a distal end of the support 566.

In use, the elongate body 530 can be extended into an intervertebral disc as previously described and moved (e.g., rotated) such that the cutting member 538 can loosen or tear tissue within a nucleus of the intervertebral disc. Tissue can simultaneously be suctioned through the internal passageway 536 via the opening 546 and/or the opening 568. The elongate body 530 can be used in with a cannula, as described in previous embodiments. In such an embodiment, the cutting member 538 can be collapsible such that as the elongate body 530 is pulled through the cannula, the cutting member 538 can collapse to a size that can fit within a lumen of the cannula. The cutting member 538 can be constructed, for example, with one or more flexible metals, flexible metal alloys, flexible shape-memory materials, and/or flexible polymeric materials.

A medical device 610 illustrated in FIG. 13 is similar to the medical device 510 shown in FIG. 12. In this embodiment, an elongate body 630 having an internal passageway 636 coupled to a source of suction power (not shown), includes a cutting member 638 also in the form of a loop. The elongate body 630 also includes an extension arm 670 extending from a distal portion 634 of the elongate body 630. In this embodiment, an opening 646 is in communication with the internal passageway 636. As with the medical device 510, the elongate body 630 can be extended through a cannula (not shown) such that the cutting member 638 is positioned within a nucleus of the intervertebral disc. The elongate body 630 can be rotated to loosen or tear tissue within the nucleus of the disc. The extension arm 670 helps direct tissue into the opening 646 as the tissue is loosened by the cutting member 638 and/or direct the extent to which the cutting member 638 rotates in a given range of directions. Simultaneously, the suction force can suction tissue through the opening 646 and through the internal passageway 636. As with the previous embodiment, the elongate body 630 can be used with a cannula. For example, the cutting member 638 can be constructed with a flexible shape memory material that allows the cutting member 638 to collapse as the elongate body 630 is pulled or pushed into a lumen of a cannula.

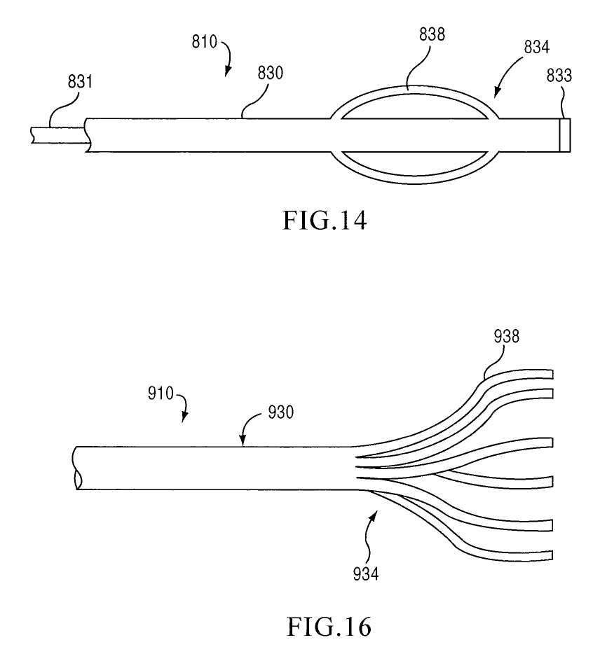

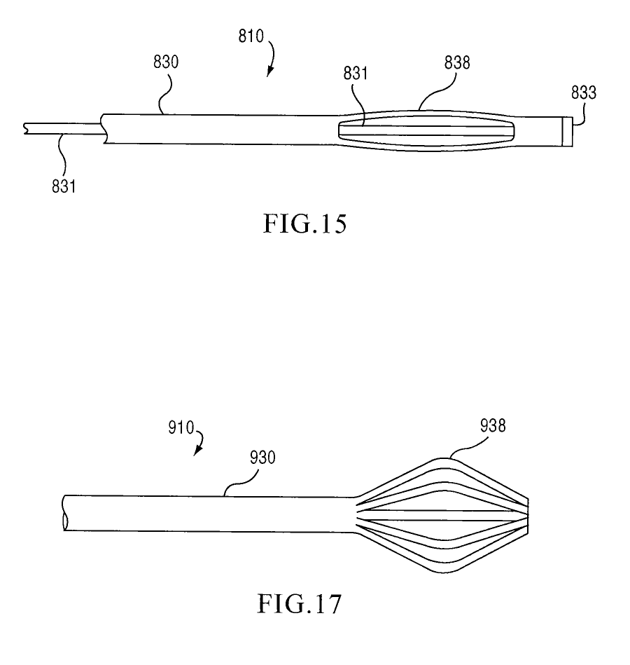

FIGS. 14 and 15 illustrate a portion of a medical device according to another embodiment of the invention. A medical device 810 includes an elongate body 830 having a distal portion 834, a proximal portion (not shown), and an internal passageway (not shown) extending from the distal portion 834 to the proximal portion. The internal passageway can be coupled to a source of suction or vacuum power (not shown) as described in the previous embodiments. The elongate body 830 includes one or more expandable cutting members 838. The cutting members 838 can be formed by cutting slits or openings along a sidewall of the elongate body 830. An actuating member can be coupled to the elongate body 830 and configured to move the elongate body 830 from a collapsed configuration as shown in FIG. 15 to an expanded configuration as shown in FIG. 14. The actuating member can be, for example, a pull rod 831 coupled to a cap 833. Other known actuating members or techniques can alternatively be used. As with the previous embodiments, the elongate body 830 can be inserted through a cannula (not shown) to percutaneously access the interior region of an intervertebral disc.

In use, with a cannula providing percutaneous access to the interior nucleus of an intervertebral disc, the elongate body 830 can be inserted through the cannula in the collapsed configuration until the distal portion 834 having the cutting members 838 is disposed within the nucleus and extending distally from the cannula. The elongate body 830 can then be moved to the expanded configuration by actuating the pull rod 831. Once in the expanded configuration, the elongate body 830 can be moved by rotating or moving the elongate body 830 in a back-and-forth manner. As the elongate body 830 is moved, the cutting members 838 can loosen tissue within the nucleus of the intervertebral disc. In some embodiments, tissue can be suctioned through the elongate body 830 as described in the previous embodiments.

In some embodiments, tissue can be captured within the openings produced by the expansion of the cutting members 838 and/or within the internal passageway of the elongate body 830. The elongate body 830 can then be collapsed, trapping the tissue within the openings of the elongate body 830. The elongate body 830 can then be removed from the intervertebral disc via the cannula.

A medical device 910 is illustrated in FIGS. 16 and 17. The medical device 910 includes an elongate body 930 having a distal portion 934, a proximal portion (not shown), and an internal passageway (not shown) extending from the distal portion 934 to the proximal portion. The internal passageway can be coupled to a source of suction or vacuum power as described in the previous embodiments. Similar to the previous embodiment, the elongate body 930 includes one or more expandable cutting members 938. The cutting members 938 can be formed by cutting the distal portion 934 into separate predefined sections. For example, the cutting members 938 can be made from a shape-memory metal that takes a predefined shape in an expanded configuration. Alternatively, the cutting members 938 can be made of a temperature-sensitive material that allow the cutting members 938 to assume an expanded configuration when exposed to certain temperatures. The elongate body 930 is configured to move from a collapsed configuration when the cutting members 938 are restrained, as shown in FIG. 17, to an expanded configuration when the cutting members 938 are unrestrained and allowed to assume their preset form, as shown in FIG. 16.

As with the previous embodiments, the elongate body 930 can be inserted through a cannula (not shown) to percutaneously access the interior region of an intervertebral disc. In use, the elongate body 930 can be inserted through the cannula in the collapsed configuration until the distal portion 934 having the cutting members 938 is disposed within the nucleus and extending distally from the cannula. Unlike the previous embodiment, the elongate body 930 assumes the expanded configuration without the aid of an actuating member, due to the cutting members 938 assuming their predefined form when released from the restraint of the cannula. In the expanded configuration, the elongate body 930 can be moved by rotating or moving the elongate body 930 in a back-and-forth manner. As the elongate body 930 is moved, the cutting members 938 can loosen or disrupt tissue within the nucleus of the intervertebral disc. In some embodiments, tissue can be suctioned through the elongate body 930 as described in the previous embodiments. In some embodiments, tissue can be captured within the space created by the cutting members 938. The elongate body 930 can then be pulled axially back into the cannula, which collapses the cutting members 938, trapping the tissue within cutting members 938. The elongate body 930 can then be removed from the intervertebral disc via the cannula.

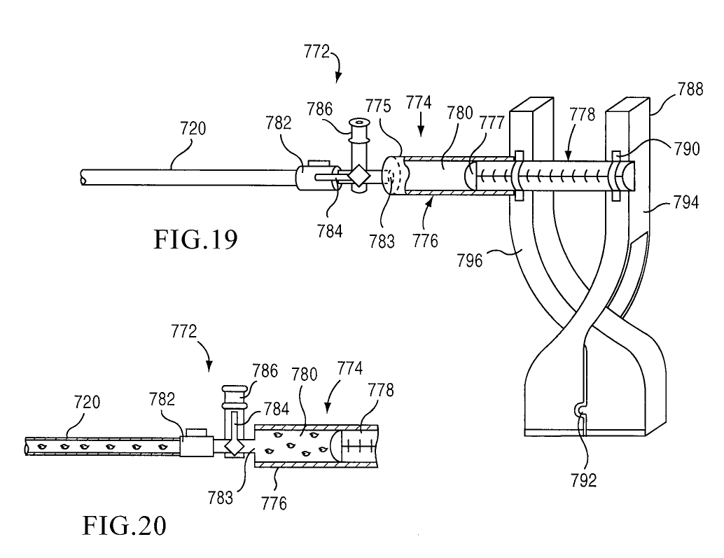

FIGS. 19 through 22 illustrate yet another embodiment of the invention. A medical device 772 includes a syringe assembly 774 having an outer elongate body 776 and an inner elongate body 778 movably disposed within a lumen 780 defined by the outer elongate body 776. The outer elongate body 776 defines an opening 783 in communication with the lumen 780. The syringe assembly 774 is coupled to a valve 782, such as a stop-cock valve so that the opening 783 is in communication with the valve 782. A lever 784 is coupled to the valve 782 to actuate the valve 782 between a closed configuration and an open configuration. An air-release valve 786 can also be coupled to the valve 782. The valve 782 can be coupled to a device configured to percutaneously access the interior of an intervertebral disc, such as a cannula 720. Cannula 720 can be, for example, any of the previously described embodiments for a cannula or any other suitable access device.

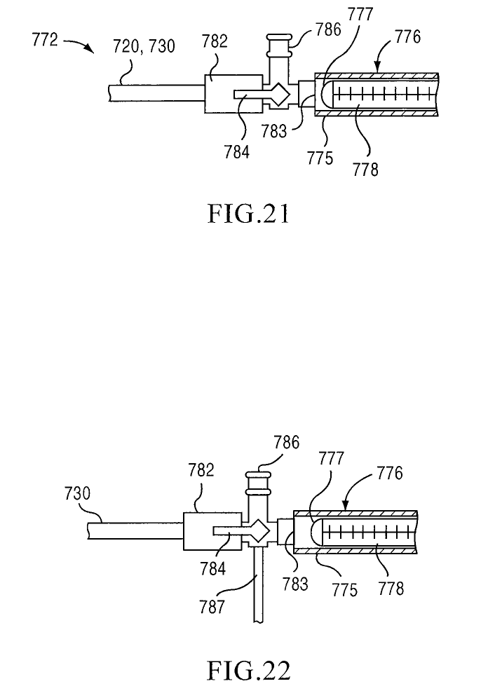

The syringe assembly 774 is coupled to a handle assembly 788 via straps 790 or other known coupling methods. The handle assembly 788 includes a first arm 794, a second arm 796, and a locking mechanism 792. Locking mechanism 792 can be, for example, a snap fit, friction fit, latch mechanism, or any other suitable locking mechanism. The handle assembly 792 can be used to move the inner elongate body 778 within the lumen 780. When a bottom portion of the first arm 794 and a bottom portion of the second arm 796 are move apart from each other, a distal end portion 777 of the inner elongate body 778 will be positioned substantially at a distal end portion 775 of the outer elongate body 776, placing the syringe assembly 774 in a closed configuration (see FIG. 21). The process of moving the syringe assembly 774 to a closed configuration includes dispelling air from within the lumen 780. The air-release valve 786 can assist with the removal of air within the lumen 780 by providing an one-way exit to dispel the air from within the syringe assembly 774.

When the first arm 794 and the second arm 776 are moved together as shown in FIG. 19, the distal end portion 777 of the inner elongate body 778 will be positioned a distance away from the distal end portion 775 of the outer elongate body 776 in an open configuration. The locking mechanism 792 can be used to secure the syringe assembly 774 in the open configuration.

The medical device 772 can be used to manually provide vacuum or suction power to the cannula 720 so that tissue can be removed from an interior of an intervertebral disc, similar to the procedures previously described. In use, the syringe assembly 774 is coupled to the valve 782 in the closed configuration, either before or after the cannula 720 has been percutaneously inserted into the intervertebral disc. The lever 784 is actuated to close the valve 782. The handle assembly 788 is actuated to move the syringe assembly 774 to the open configuration as shown in FIG. 19. The process of moving the syringe assembly 774 into the open configuration creates a vacuum condition within the lumen 780 of the outer elongate body 776. The lever 784 can then be moved such that the valve 782 is opened. This will allow the vacuum pressure within the syringe assembly 774 to suction or vacuum tissue from the nucleus of the intervertebral disc, through the cannula 720 and into the lumen 780 as shown in FIG. 16. The lever 784 can then be actuated to shut the valve 782. To remove the tissue from the lumen 780, the syringe assembly 774 can be decoupled from the valve 782. The tissue held within the lumen 780 can be dispelled from the syringe assembly 774 through the opening 783 and into a container or other disposal means, by actuating the handle assembly 788 to move the syringe assembly 774 into the closed configuration, as shown in FIG. 21.

In alternative embodiments, the medical device 772 can be used in conjunction with any of the embodiments of an elongate body described above, to manually provide suction or vacuum pressure to the internal passageway of the elongate body. In such an embodiment, the medical device 772 can be coupled to an elongate body 730 and provide a means to dispel the tissue from within the lumen 780 without having to disengage the syringe assembly 774 from the valve 782. This would allow the above described method of using the medical device 772 to be repeated as necessary to produce repeated vacuum forces within the internal passageway of the elongate body 730. For example, the valve 782 can be coupled to a transfer line 787, through which the tissue can be dispelled from the syringe assembly 774 as shown in FIG. 22. In such an embodiment, the valve 782 can include a first mode of actuation in which the valve is closed to both the elongate body 730 and the transfer line 787, a second mode of actuation in which the valve is open to the elongate body 730, but closed to the transfer line 787, and a third mode of actuation in which the valve is closed to the elongate body 730, but open to the transfer line 787.

In another variation of the above embodiment, instead of the valve 782 being coupled to the transfer line 787, a second stop valve (not shown) can be coupled to the syringe assembly 774. The second stop valve can be opened to allow the tissue within lumen 780 to be dispelled in a similar manner as described above.

The medical device for any of the embodiments may be constructed with any suitable material used for such a medical device. For example, the elongate body and the cannula can be constructed with a biocompatible material, such as stainless steel or suitable plastic materials such as various polymers. The cutting member can likewise be constructed with suitable biocompatible metals or plastics.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

For example, in some embodiments, a cannula may or may not be included in the medical device. In any of the embodiments, the elongate body can include means to irrigate via one or more irrigation lumens. In some embodiments, a handle assembly is included and an actuation trigger coupled to the handle. In some embodiments, means for manually providing suction or vacuum power can be used as described in FIGS. 19-22, rather than a battery or other automated power source. In some embodiments the elongate body and/or a tissue containment device can be constructed with a translucent material, so that the physician can view the tissue being suctioned through the internal passageway and into the containment device.

What is claimed is:

1. An apparatus, comprising:
an elongate body having a collapsed configuration and an expanded configuration,
the elongate body having a cutting portion at a distal portion of the elongate body, the cutting portion defining an opening in the elongate body,
the distal portion of the elongate body configured to be inserted into a nucleus of an intervertebral disc when the elongate body is in the collapsed configuration, the cutting portion of the elongate body configured to disrupt at least a portion of tissue within the nucleus of the intervertebral disc when the elongate body is in the expanded configuration,
the elongate body configured to capture the portion of tissue within the opening of the elongate body when the elongate body is moved from the expanded configuration to the collapsed configuration while disposed within the nucleus of the intervertebral disc.

2. The apparatus of claim 1, further comprising:
a cannula defining a lumen, the elongate body configured to be movably disposed within the lumen of the cannula, the elongate body is formed with a shape-memory material such that when the elongate body is moved from within the lumen of the cannula to a location outside of the lumen of the cannula, the elongate body is moved from the collapsed configuration to the expanded configuration.

3. The apparatus of claim 1, further comprising:
an actuating member coupled to the elongate body, the actuating member configured to move the elongate body between the collapsed configuration and the expanded configuration.

4. The apparatus of claim 1, further comprising:
a pull rod coupled to the elongate body, the pull rod configured to move the elongate body between the collapsed configuration and the expanded configuration.

5. The apparatus of claim 1, further comprising:
a cannula defining a lumen,
the elongate body configured to be movably disposed within the lumen of the cannula when the elongate body is in the collapsed configuration,
the distal portion of the elongate body having a size while in the expanded configuration greater than a size of the lumen of the cannula.

6. The apparatus of claim 1, wherein the elongate body defines an internal passageway defined, the opening of the elongate body is in communication with the internal passageway, the internal passageway configured to be coupled to a suction source, and.
when the portion of tissue is captured in the opening of the elongate body and the suction source is activated, the portion of tissue is removed through the internal passageway of the elongate body.

7. The apparatus of claim 1, wherein the cutting portion is a first cutting portion, the elongate body includes a plurality of cutting portions, the plurality of cutting portions includes the first portion, each cutting portion from the plurality of cutting portions having a curved cross-section.

8. The apparatus of claim 1, wherein the distal portion of the elongate body has a plurality of cutting portions including the cutting portion, the plurality of cutting portions formed by cutting longitudinal slits in the outer wall of the elongate body.

9. The apparatus of claim 1, wherein the cutting portion of the elongate body is configured to disrupt at least a portion of the tissue when the elongate body is rotated within the nucleus of the intervertebral disc.

10. An apparatus, comprising:
a cannula defining a lumen; and
an elongate body having a collapsed configuration and an expanded configuration, the elongate body configured to be movably disposed within the lumen of the cannula when the elongate body is in the collapsed configuration,
the elongate body having a cutting portion at a distal portion of the elongate body, the cutting portion defining an opening in the elongate body,
the distal portion of the cannula configured to be inserted into an interior portion of an intervertebral disc when the elongate body is in the collapsed configuration,
the cutting portion of the elongate body configured to disrupt at least a portion of tissue within the interior of the intervertebral disc when the distal portion of the elongate body is moved outside of the lumen of the cannula and within the interior of the intervertebral disc while the elongate body is in the expanded configuration, the elongate body configured to capture the portion of tissue within the opening of the elongate body when the elongate body is moved from the expanded configuration to the collapsed configuration.

11. The apparatus of claim 10, wherein the elongate body is retracted into the distal portion of the cannula when the elongate body is moved between the expanded configuration and the collapsed configuration.

12. The apparatus of claim 10, further comprising:
an actuating member coupled to the elongate body, the actuating member configured to move the elongate body between the collapsed configuration and the expanded configuration.

13. The apparatus of claim 10, further comprising:
a pull rod coupled to the elongate body, the pull rod configured to move the elongate body between the collapsed configuration and the expanded configuration.

14. The apparatus of claim 10, wherein the elongate body defines an internal passageway, the opening in the elongate body is in communication with the internal passageway, the internal passageway is configured to be coupled to a vacuum source; and
when the portion of tissue is captured in the opening of the elongate body and when the vacuum source is activated the portion of tissue is removed through the internal passageway of the elongate body.

15. The apparatus of claim 10, wherein the cutting portion is a first cutting portion,
the distal portion of the elongate body includes a plurality of cutting portions, the plurality of cutting portions includes the first cutting portion.

16. The apparatus of claim 10, wherein the distal portion of the elongate body has a plurality of cutting portions including the cutting portion, the plurality of cutting portions formed by cutting longitudinal slits in the outer wall of the elongate body.

17. The apparatus of claim 10, wherein the cutting portion of the elongate body is configured to disrupt at least a portion of tissue when the elongate body is moved within the interior portion of a the intervertebral disc while in the expanded configuration.

18. The apparatus of claim 10, wherein the elongate body is formed with a shape-memory material such that when the elongate body is moved from within the lumen of the cannula to a location outside of the lumen of the cannula, the elongate body is moved from the collapsed configuration to the expanded configuration.

19. A method, comprising:
percutaneously inserting an elongate body in a collapsed configuration at least partially into a nucleus of an intervertebral disc, the elongate body having a cutting portion at a distal portion of the elongate body, the cutting portion defining an opening in the elongate body;
expanding the elongate body from the collapsed configuration to an expanded configuration while disposed within the nucleus of the intervertebral disc;
rotating the elongate body during the expanding such that the cutting portion disrupts at least a portion of tissue within the nucleus of the intervertebral disc; and
collapsing the elongate body from the expanded configuration to the collapsed configuration to capture at least a portion of the disrupted tissue within the opening of the elongate body.

20. The method of claim 19, further comprising:
activating a suction source coupled to the elongate body such that at least a portion of the disrupted tissue is removed from the intervertebral disc through the lumen of the elongate body.

21. The method of claim 19, wherein the expanding the elongate body includes actuating a pull rod.

22. The method of claim 19, wherein:
the expanding the elongate body to the expanded configuration includes moving a cannula in which the elongate body is movably disposed such that the distal portion of the elongate body is disposed outside of the cannula and within the nucleus of the intervertebral disc.

23. The method of claim 19, wherein:
wherein collapsing the elongate body to the collapsed configuration includes moving a cannula in which the elongate body is movably disposed such that the distal portion of the elongate body is disposed within the cannula.
